(19) United States
(12) Patent Application Publication
(10) Pub. No.: US 2001/0053772 A1
(43) Pub. Date: Dec. 20, 2001

(54) Aza analogues of alkyl lysophospholipids exert immunomodulatory effects

(76) Inventors: Benjamin Bonavida, Los Angeles, CA (US); Xiao-Hu Gan, Granada Hills, CA (US)

Correspondence Address: MILORD & ASSOCIATES, 2029 CENTURY PARK EAST, SUITE 1700, LOS ANGELES, CA, 90067

(21) Appl. No.: 09/846,466

(22) Filed: Apr. 30, 2001

Related U.S. Application Data
(60) Provisional application No. 60/200,822, filed on Apr. 28, 2000.

(51) Int. Cl. A61K 31/685

(52) U.S. Cl. 514/78

(57) ABSTRACT

The present invention relates to alkyl lysophospholipid (ALP), and derivatives thereof, that are potent cytokine inducers and immunomodulatory agents. The immunomodulatory agents are administered to a subject having a condition characterized by an altered or aberrant cytokine activity such as, but not limited to, neoplasia, infectious diseases, chronic and acute immune diseases, autoimmunity, transplantation, diseases mediated by nitric oxide and cytokines, adverse drug reactions, obesity, septic shock, and adverse side effects due to cancer chemotherapy.

### RELATED APPLICATION

This application claims priority from U.S. provisional patent application No. 60/200,822 filed on Apr. 28, 2000 and entitled “AZA ANALOGUES OF ALKYL LYSOPHOSPHOLIPIDS EXERT IMMUNOMODULATORY EFFECTS,” the contents of which are incorporated herein by reference.

### FIELD OF THE INVENTION

The present invention relates generally to the fields of phospholipids and molecular immunology and, more specifically, to novel cytokine immunomodulatory agents and their use in controlling cytokine-regulated physiologic processes and pathologies.

### BACKGROUND OF THE INVENTION

Among the most successful biotechnology drugs that have reached the market are a variety of cytokines and cytokine modulators such as PROTROPIN NUTROPIN, INTRON A ROFERON-A, AVONEX BETASERON, ACTIMMUNE, EPOGEN, NEUPOGEN, LEUKINE, NEUMEGA, PROLEUKIN REMICADE, ENBREL, and HERCEPTIN. The isolation and characterization of genes encoding cytokines and their receptors has greatly facilitated the analysis of cytokine function and application to disease. However, all of the aforementioned medicines are recombinant proteins and as such they suffer from a number of potential drawbacks. These may include difficulty and expense of synthesis, a requirement for parenteral administration, and the potential for immune responses. Accordingly, it is more effective to use non-peptidyl cytokine modulators to treat the same because of the ease of manufacture thereof, they are bioavailable, and non-immunogenic.

Cytokines are a class of secreted, soluble proteins produced by a variety of cells in response to many different kinds of inducing stimuli, including environmental, mechanical, and pathological stresses. Lymphoid, inflammatory and hemopoietic cells secrete a variety of cytokines which regulate the immune response by controlling cell proliferation, differentiation and effector functions. For example, regulatory cytokines produced in response to T cell stimulation during an immune response can be immunosuppressive or immunostimulatory. The immune response and acute phase response associated with altered cytokine levels can occur, for example, due to disuse deconditioning, organ damage such as that associated with transplantation, cancer treatment, septic shock and other bacterially related pathologies, adverse drug reactions, nitric oxide mediated tissue damage and diabetes.

Cytokines are normally present in very low concentrations in a tissue and their effects are mediated through binding to high affinity receptors on specific cell types. Various cytokines such as the interleukins (IL), interferons (IFN), colony stimulating factors (CSF) and tumor necrosis factors (TNF) are produced during immune, inflammatory, repair and acute phase responses and they control various aspects of these responses. Following induction of such an immune, inflammatory, repair or acute phase response, the concentrations of various cytokines can increase or decrease at different times. For example, increased levels of cytokines are associated with a variety of situations such as space flight, immobilization, spinal cord injury, and bed rest, which result in disuse deconditioning. During space flight, for example, TNF, IL-6, and IL-2 levels increase upon a subjects initial exposure to weightlessness and again upon return from space.

Altered levels of cytokines have also been linked to abnormal bone metabolism and the rapid decalcification that occurs during immobilization, spinal cord injury, or long-term bed rest. Similarly, cytokine levels are altered during chronic states such as during repair and autoimmune reactions to organ damage, nephrotoxicity associated with the administration of cyclosporine to transplant subjects, cancer chemotherapy, as well as in individuals that are obese or suffering from diabetes, septic (endotoxic) shock or glomerulonephritis.

Cytokines, including the TNFs, CSFs, interferons and interleukins mediate host defense responses, cell regulation and cell differentiation. For example, these cytokines can induce fever in a subject, can cause activation of T cells, B cells and macrophages, and can affect the levels of other cytokines, which result in a cascade effect whereby other cytokines mediate the biological levels and actions of the first cytokine.

Cytokines may regulate the immune response through immunostimulatory or immunosuppresive effects. For example, IL-10 can block activation of many of the inflammatory cytokines including TNF, IL-1 and IL-6, while upregulating anti-inflammatory cytokines, such as IL-12. IL-10, which is produced by macrophages and other cell types, also stimulates the proliferation of mast cells and thymocytes and inhibits various functions of monocytes and macrophages. As a consequence of this monocyte and macrophage inhibition, the activity of T cells is also affected. The full scope of the role of IL-10 in the immune system is only beginning to be understood.

Cytokines have multiple biological activities and interact with more than one cell type. Thus, it has not been possible to target one particular cytokine or cell type to prevent the damaging side effects of treatment. A better approach for preventing damage due to the unwanted and uncontrolled over-suppression or over-stimulation of cytokine activity would be to regulate the expression of the relevant or controlling cytokine or cytokines involved in an immune response without eliminating or over-expressing any one cytokine. Such a treatment would not create or aggravate a pathological or ongoing immune response. In this way, pathological immune-mediated effects, such as immunosuppression or autoimmune reactions, can be prevented and homeostasis can be maintained.

Corticosteroids can be used to modulate cytokine expression. However, they can cause complete immunosuppression and have other undesirable side effects, such as inducing “wasting” syndrome, diabetes and osteoporosis. Similarly, non-steroidal anti-inflammatory drugs (NSAID), such as ketorolac (Toradol.RTM.; Syntex), are effective in treating inflammation and pain. However, NSAIDs also cause undesirable side effects by inhibiting prostaglandin production, which can lead to potentially severe complications including gastric ulceration, bleeding and renal failure.

In order to prevent pathological conditions or disruption of normal immune mediated functions caused by the aberrant expression of cytokines as described above, it would be advantageous if cytokine levels could be accurately manipulated and efficaciously controlled. Thus, a need exists for agents that can regulate the activity of cytokines in a subject without causing undesirable side effects. Furthermore, a need exists for identifying agents which can be used in the treatment of pathologies and conditions associated with altered cytokine levels. The present invention satisfies these needs and provides related advantages as well.

### SUMMARY OF THE INVENTION

Accordingly, the primary objective of the present invention is to overcome the limitations of the prior art.

Another object of the invention is to provide methods for treating individuals having at least a disease by the use of synthetic aza alkyl lysophopholipids and analogues thereof that are cytokine immunomodulatory agents.

It is another object of the present invention to provide at least a pharmaceutical composition comprising a pharmaceutically acceptable carrier and at least a cytokine immunomodulatory agent.

It is another object of the present invention to provide a method for administering such a cytokine regulatory agent or a combination thereof to a subject to modify the levels of various cytokines in the individual, which will result in immunostimulation or immunosuppression depending on the particular agent administered.

It is a further object of the present invention to provide a method for regulating through enhancement the cytokine activity in a subject and methods of treating a condition, pathology or an injury characterized, in part, by altered or aberrant cytokine activity. Such conditions, pathologies or injuries include, but are not limited to, disuse conditioning, diseases mediated by ctyokine or nitric oxide, diabetes, obesity, autoimmune diseases, septic (endotoxic) shock, glomerulonephritis, organ damage such as that which occurs during transplantiation and adverse side effects of cancer chemotherapy, such as nephrotoxicity. In addition, the present invention provides methods to enhance the effectiveness and potency of various immunotherpeutic interventions as well as the response to vaccines.

Such stated objects and advantages of the invention are only examples and should not be construed as limiting the present invention. These and other objects, features, aspects, and advantages of the invention herein will become more apparent from the following detailed description of the embodiments of the invention when taken in conjunction with the accompanying figures and the claims that follow.

### BRIEF DESCRIPTION OF THE FIGURES

It is to be understood that the drawings are to be used for the purposes of illustration only and not as a definition of the limits of the invention.

FIG. 1. This figure illustrates the chemical structure of five aza-alkyl-lysophospholipids under each of the compound name, the molecular weight (mw) is listed in parentheses.

FIG. 2. illustrates a table wherein Peripheral Blood Mononuclear (PBM) cells were incubated for 18 h at 37° C. in the absence or in the presence of different concentrations of AALP analogues. The cell-free supernatants were harvested and clarified by centrifugation and frozen at −70° C. until further use. The supernatants were assessed for the presence of TNF-α, IL-1β, and IL-6 by ELISA. The numbers represent the mean of duplicate wells.

FIG. 3. Induction of cytokine secretion by BN52207. Human PBM were incubated for 18 h at 37° C. in the presence or absence of different concentrations of BN52207. The PBM were left untreated or treated with IFN-γ (500 μg/ml) or LPS (1 μg/ml) added at the initiation of the culture. The supernatants were harvested, clarified by centrifugation, and stored at −70° C. The levels of TNF-α with IFN-γ (A), LPS (B), or BN+LPS (C) activated PBM were assessed by ELISA. *P<0.01. The synthesis of TNF-α by BN52207 was monitored by RT-PCR. For RNA extraction, 2×10⁶ cells were used for each sample. Monocytes were incubated with BN, with or without LPS (1 μg/ml), for 3 h. The total cellular RNA was extracted by the guanidinum-isothiocyanate/phenol method. Total RNA (250 μg/sample) was used for RT-PCR, and PCR products (25 cycles) were stained by ethidium bromide (D). (Top) Photograph of gel. (Bottom) The negative film was scanned by laser densitometr (LKB2222-020 ultrascan XL, Pharmacia). The percentage surface area under the peak of each band for TNF-α mRNA was normalized to the corresponding GAPDH band.

FIG. 4. Inhibition of TNF-α secretion. PBM treated with protein synthesis inhibitors. PBM were incubated for 18 h with BN52207 (5 μg/ml) in the absence or presence of LPS (1 μg/ml)+PTX (50 μg/ml), emetine (1 μg/ml), and cycloheximide (5 μg/ml). The cell-free supernatants were collected and clarified by centrifugation and stored at −70° C. until use. The level of TNF-α in the supernatants was assessed by ELISA.

FIG. 5. Induction of IL-1β secretion by BN52207. PBM were incubated for 18 h at 37° C. with different concentrations of BN52207 in the absence or presence of rIFN-γ (1000 U/ml) (A) or LPS (1 μg/ml). (B) The supernatants were harvested, clarified by centrifugation, and stored at −70° C. until use. IL-1β was assessed by ELISA.

FIG. 6. The synthesis of TNF-α, IL-1β and IL-10 was monitored by RT-P CR. (A) Photograph of gel. (B) The percentage surface area under the peak of each band was normalized to the corresponding GADPH band.

FIG. 7. Inhibition of IL-10 secretion by BN52207. PBM were incubated for 72 h at 37° C. in the presence of BN52207 (5 μg/ml) and in the absence or presence of LPS (1 μg/ml). At different times after culture, supernatant aliquots were removed and clarified by centrifugation and stored at −70° C. until used. The level of IL-10 was assessed by ELISA.

FIG. 8. illustrates the kinetics of cytokine secretion by medium, BN52207, LPS, or LPS and BN52207 on PBM. At different time intervals after initiation of cultures, aliquots from supernatants were removed and stored. The presence of TNF-α (A), IL-1β (B), and IL-10 (C) was assessed by ELISA.

FIG. 9. illustrates the induction of cytotoxicity by BN-treated PBM. PBM were cultured for 18 h at 37° C. in the presence of various concentrations of BN52207 and in the absence or presence of rIFN-γ (1000 U/ml). After one washing the cells were harvested and assayed for cytotoxicity against ⁵¹Cr-labeled U937 targets in an 18 h cytotoxicity assay. Cytotoxicity was calculated in LU/10⁶ cells from the E:T ratios used. One LU/10 represents a specific cytotoxiciy of 10% by 10⁶ effector cells.

### DETAILED DESCRIPTION OF THE INVENTION

This invention relates to the immunomodulatory and sensitizing effects of lysophospholipids in general, and specifically relates to the immunomodulatory effects of analogues of Aza Alkyl Lysophospholipids (AALP). The present invention also provides methods of regulating, through enhancement of the cytokine activity in a subject, and methods of treating a condition, pathology or an injury characterized, in part, by altered or aberrant cytokine activity.

In addition, the present invention relates to pharmaceutical compositions comprising a pharmaceutically acceptable carrier and a cytokine regulatory agent or agents. Administration of such a cytokine regulatory agent or a combination thereof to a subject can modify the levels of various cytokines in the individual, which will result in immunostimulation or immunosuppression depending on the particular cytokines that are regulated.

Furthermore, the present invention allows treatment of a variety of drug resistant neoplasms or diseased tissue by the combination of cytotoxic and non-cytotoxic agents. In addition, the combination of the immunomodulatory factors and cytotoxic agents allows the use of a decreased concentration of cytotoxic agents while increasing the pharmacosensitivity of the neoplasm or diseased tissue to such treatment. In particular, the present invention utilizes a combination of agents to increase the sensitivity of tumor cells to the cytotoxic effects of the agents and eventual destruction thereof, by necrosis or apoptosis for example. Cancer cells from numerous tumor cell lines of various origin are susceptible to the cytotoxic affects of the present invention, including, but not limited to, drug and immune resistant cancer cells and fresh tumor cells. Throughout this specification, the term “tumor” is understood to mean cancer cells or a collection of cancer cells, whether in solid or suspension form. Although, hereinafter, a variety of preferred embodiments are disclosed, these should not be construed as limitations on the scope of the invention.

Throughout this specification, the term “drug” or “agent” is understood to mean any substance used in the diagnosis, treatment, cure, and prevention of diseases, such as but not limited to cancer or cell proliferative disorder. More specifically, the term “drug” or “agent” will be used to apply to substances which are known or suspected to directly or indirectly prevent or inhibit the growth of cancer cells, either by directly attacking the cancer cells, by stimulating the body's immune system, or by other means. It is understood that “drug” or “agent” also encompasses substances which are not believed to prevent or inhibit the growth of cancer cells by themselves, but which are known or suspected to do so when used in combination with one or more other drugs. The term “drug” or “agent” includes, but is not limited to, chemotherapeutic drugs recognized by the prior art, as well as the biological response modifiers described below, alkylating agents, antibiotics, antimetabolites, and palliatives or drugs used to combat the side effects of chemotherapy.

Biological response modifiers are substances whose origin is endogenous, or which are man-made and mimic particular biological functions of such substances. Biological response modifiers have a synergistic effect and produce increased tumor cell killing with a number of chemotherapeutic agents, have antitumor activity of their own, enhance populations of immune effector cells, such as natural killer cells, monocytes and cytotoxic T cells, and lower levels of tumor growth factors. These biological response modifiers (BRM) are collectively known as cytokines, immuno-adjuvants, or immuno-modulators. These BRM include, but are not limited to, interferons, interleukins, tumor necrosis factors, transforming growth factors, granulocyte-colony stimulating factor, granulocyte macrophage-colony stimulating factor, other cytokines and growth factors, and synthetic or recombinant modified molecules. For example, interferons are closely related glycoproteins with antiviral, immunoregulatory and antiproliferative functions. The immunoregulatory functions of histocompatibility antigens, the activation of monocytes and or macrophages, and T and B cell functions have proven to be of clinical importance.

As used herein, the terms “Immunomodulate” or “immunomodulatory” mean to control by enhancing, limiting, restricting, restraining, modulating or moderating both the synthesis and secretion. Such regulation includes pleiotropic, redundant, synergistic or antagonistic effects that occur due to the activity of biological agents such as cytokines, which can affect a variety of biological functions directly or indirectly through cascade or biofeedback mechanisms.

As used herein, the term “cytokine immunomodulatory agent” means an agent that controls cytokine activity by enhancing, limiting, restricting, restraining, modulating or moderating the biological activity of a cytokine. It should be recognized, however, that while the cytokine regulating agents generally can regulate cytokine activity, no specific mechanism of action is proposed as to how a cytokine regulatory agent acts to effect a condition characterized by altered or aberrant cytokine activity.

Cytokines are well known in the art and include, but are not limited to the tumor necrosis factors (TNFs), colony stimulating factors (CSFs), interferons (INFs), interleukins (IL-1, IL-2, IL-3, IL-4, IL-5, IL-6, IL-7, IL-8, IL-9, IL-10, IL-11, IL-12, IL-13, IL-14, and IL-15), transforming growth factors (TGFs), oncostatin M (OSM), leukemia inhibiting factor (LIF), platelet activating factor (PAF) and other soluble immunoregulatory peptides that mediate host defense responses, cell regulation and cell differentiation (see, for example, Kuby, Immunology 2d ed. (W. H. Freeman and Co. 1994); see Chapter 13, which is incorporated herein by reference).

The cytokine regulatory agents of the invention can regulate the aberrant or altered expression of one or more cytokines that occurs in various conditions, including, for example, pathologies, immune responses and inflammatory responses. Such conditions are considered together for purposes of the present invention is that they are characterized, in part, by altered or aberrant cytokine activity and, therefore, are amenable to regulation by one or more cytokine regulatory agents.

As used herein, the term “characterized by” means contributes or affects, at least in part. Though cytokine contribution can be, it does not have to be, and the only, primary, or even a major factor of the condition. For example, it is well understood in the art that an infection has altered cytokine levels and is, therefore, a condition characterized by cytokine activity, but that cytokines activity is only a part of the infectious condition.

As used herein, the term “condition characterized by altered or aberrant cytokine activity” includes all cytokine regulated or modulated pathologies and injuries, including the immune, inflamatory and healing processes associated with an injury. The skilled artisan can recognize such a condition by detecting an increased or decreased level or activity of a particular cytokine as compared to the normal level of the cytokine expected to be found in a healthy individual. Methods for determining such normal levels are well known in the art.

Conditions characterized by altered or aberrant cytokine activity include, but are not limited to, disuse deconditioning, organ damage such as occurs in response to organ transplantation; adverse reactions associated with cancer chemotherapy; obesity; diseases such as diabetes and atherosclerosis that are mediated by free radicals and nitric oxide action; bacterial endotoxic sepsis and related shock; pain; cachexia; adult respiratory distress syndrome; and autoimmune or other patho-immunogenic diseases or reactions such as allergic reactions or anaphylaxis, arthritis, inflammatory bowel disease, glomerulonephritis, systemic lupus erythematosus, transplant atherosclerosis and parasitic mediated immune dysfunctions such as Chagas' Disease.

The invention also relates to pharmaceutical compositions comprising a cytokine regulatory agent or agents and a pharmaceutically acceptable carrier. Pharmaceutically acceptable carriers are well known in the art and include aqueous solutions such as physiologically buffered saline or other buffers or solvents or vehicles such as glycols, glycerol, oils such as olive oil or injectable organic esters.

A pharmaceutically acceptable carrier can contain physiologically acceptable compounds that act, for example, to stabilize the cytokine regulatory agent or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the cytokine regulatory agent and on the particular physio-chemical characteristics of the specific cytokine regulatory agent.

A cytokine regulatory agent as described above or a pharmaceutical composition containing a cytokine regulatory agent can be administered to a subject in order to regulate pathologically modified, including depressed or elevated, cytokine activity in the subject. For example, the peptide or composition can be administered to a subject as a treatment for traumatic injuries, bacterial sepsis and endotoxic shock, inflammation, pain, cachexia, adult respiratory distress syndrome, transplant atherosclerosis, and patho-immunogenic diseases such as arthritis, inflammatory bowel disease systemic lupus erythematosus and other autoimmune dysfunctions, each of which is characterized by pathologically elevated inflammatory cytokine activity.

As used herein, the term “pathologically elevated” means that a cytokine activity is elevated above a range of activities which is expected in a normal population of such subjects and which is associated with a pathological response. For example, a normal range of IL-1, such as IL-1β, activity present in a specific tissue can be determined by sampling a number of subjects in the population. A subject having a pathology characterized by cytokine-induced pathological effects can be readily identified by determining that the cytokine activity in the subject is pathologically elevated, which is above the normal range and which results in a pathological response such as a fever.

Cytokines also play an important role in organ damage and organ protection. For example, cytokines significantly affect such events and conditions as organ transplant, particularly the rejection of a transplanted organ, transplant atherosclerosis, ischemia-reperfusion, cyclosporine-induced nephrotoxicity, myocardial infarction, and stroke.

A cytokine regulatory agent of the present invention, or composition containing the agent, can also be used in cancer chemotherapy for reducing the nephrotoxic effect or other negative effects of cancer chemotherapeutic agents.

Cytokine regulatory agents of the present invention can be used for treating a subject having a disease mediated by nitric oxide and cytokines, such as diabetes and glomerulonephritis. As used herein, the term “treating” includes a meaning that encompasses reducing or alleviating one or more symptoms or conditions associated with a particular disease state mediated by NO and cytokines. For example, treating diabetes can be manifested by reducing glucose levels of a diabetic.

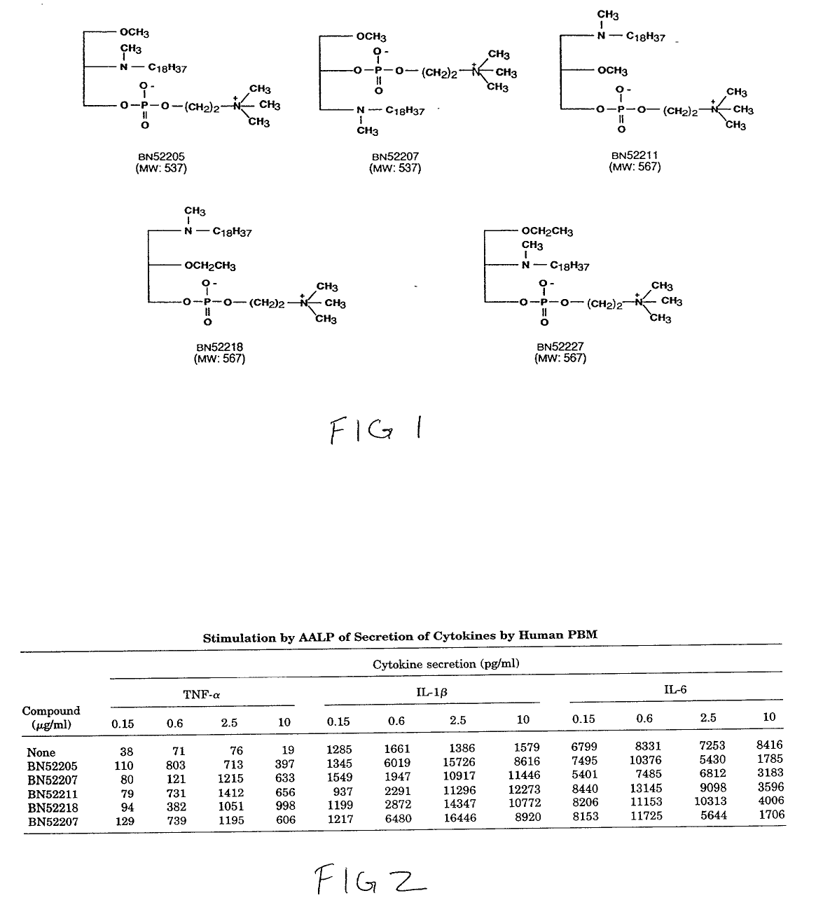

Now referring to FIG. 1, for purposes of illustration and not limitation, five AALP compounds which were synthesized at Institut Henri-Beaufour in France are therein illustrated (BN52205, BN52207, BN52211, BN52218, AND BN52227). It is to be understood that although a particular aza group and alkyl group are herein presented, the invention is not limited to the illustrated groups and can be any aza and alkyl group that provide similar results. Accordingly, the glycerol derivatives of Braquet et al. set forth in U.S. Pat. No. 5,116,992 and titled “Glycerol Derivatives, and Therapeutical Compositions Containing Them,” is incorporated herein by reference. As a result, the invention relates to glycerol derivatives of general formulae 1a, 1b, and 1c:

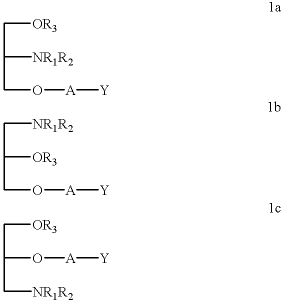

wherein R1 represents a hydrogen atom or a lower alkyl group up to C5;

R2 represents a straight chain or branched chain alkyl group having from 10 to 24 carbon atoms;

R3 represents an aryl or an alkyl radical, CONH-alkyl, CON-dialkyl, each alkyl group having from 1 to 6 carbon atoms;

A stands for:

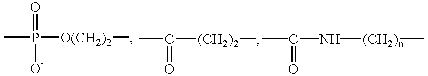

n being an integer of from 2 to 10;

Y represents the following quaternary ammonia: ammonium, alkylammonium, dialkylammonium, trialkylammonium, each alkyl group having from 1 to 6 carbon atoms, or a saturated or unsaturated heterocycle containing nitrogen atom as hetero atom.

Also referring to FIG. 2, the AALP analogues, are therein shown to have immunoregulatory properties. The effect of AALP on the secretion of TNF-α, IL-1β, IL-6, and IL-10 cytokines by both resting and LPS and IFN-γ activated human peripheral blood derived monocytes. Human PBM were incubated for 18 hours at 37° C. in the absence or in the presence of different concentrations of AALP analogues. The cell-free supernatants were harvested and clarified by centrifugation and assessed for the presence of TNF-α, IL-1β, and IL-6 by Enzyme Linked Immunosorbant Assay (ELISA). For TNF-α, there was stimulation of secretion by all compounds at concentrations >0.6 μg/ml and up to 10 μg/ml. Plateau secretion was reached at 2.5 μg/ml levels and secretion was inhibited at 10 μg/ml. Similar findings were also obtained for IL-1β. There was no significant difference in the levels of stimulated TNF-α and IL-1β among the five AALP compounds. In contrast to TNF-α and IL-1β, there was no significant stimulation of IL-6 above background levels upon stimulation by any of the five AALP analogues. However, there was significant inhibition of IL-6 secretion at 10 μg/ml and the levels were below the spontaneous secretion of IL-6.

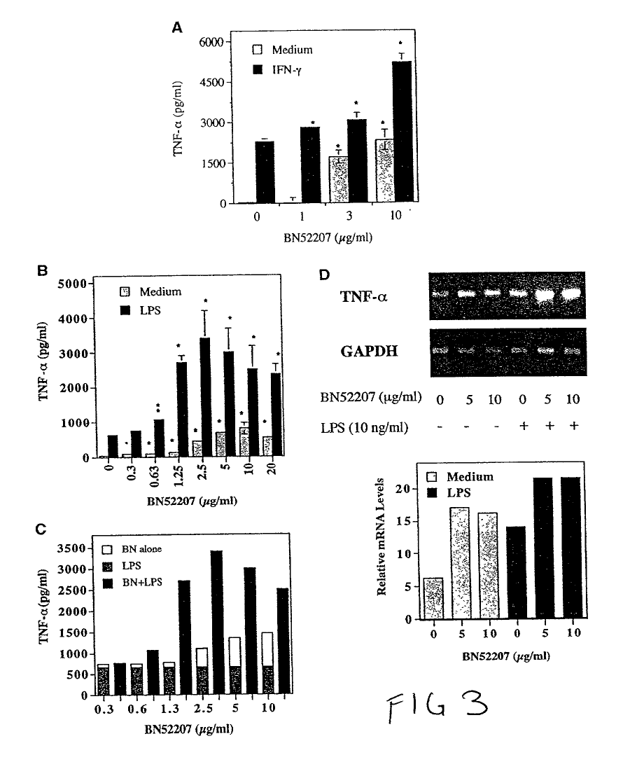

As a result of the similarity of results of the five AALP on PBM, BN52207 was selected to conduct the following experiments. However, it is to be understood that any other AALP analogue may be used to obtain a similar result. Now referring to FIG. 3, human PBM were incubated for 18 hours at 37° C. in the presence or absence of different concentration of BN52207. The PBM were left untreated or treated with IFN-γ (500 U/ml) or LPS (1 μg/ml) added at the initiation of the culture. The supernatants were harvested, clarified by centrifugation, and stored at −70° C. The levels of TNF-α with IFN-γ (FIG. 3A), LPS (FIG. 3B), or BN+LPS (FIG. 3C) activated PBM were assessed by ELISA.

In FIG. 3A, stimulation of PBM with IFN-γ results in the secretion of TNF-α and the addition of BN52207 significantly potentiated the IFN-γ mediated stimulation of TNF-α synthesis and secretion. Unstimulated cells also responded to BN52207 and secreted TNF-α at concentrations >3 μg/ml. These findings demonstrate that AALP have the capacity to stimulate both resting and activated PBM for TNF-α secretion.

In FIG. 3B, stimulating of PBM with LPS resulted in some TNF-α secretion and BN52207 potentiated the secretion of TNF-α. However, in the presence of LPS there was a significant stimulation and synergy was achieved from TNF-α secretion. In FIG. 3C the combination of LPS and BN52207 resulted in more of an additive effect and synergy was achieved from TNF-α secretion. These findings demonstrate clearly that AALP stimulates the synthesis of TNF-α in LPS stimulated PMB and the synergy achieved suggest strongly that AALP regulates the transcriptional machinery of TNF-α transcription and translation.

In addition to the secretion of TNF-α, de novo synthesis of TNF-α was determined by examining RNA transcription. The synthesis of TNF-α by BN52207 was monitored by RT-PCR. For RNA extraction, 2×10⁶ cells were used for each sample. Monocytes were incubated with BN52207, with or without LPS (1 μg/ml), for 3 hours. The total cellular RNA was extracted by the guanidinum-isothicyanate/phenol method. Total RNA (250μg/sample) was used for TNF-α mRNA by RT-PCR, and PCR products (25 cycles) were stained by ethidium bromide, as seen in FIG. 3D. Clearly, BN52207 stimulated de novo synthesis of TNF-α transcripts in both untreated and LPS treated monocytes. These findings corroborate the ELISA and demonstrated that both synthesis and secretion are induced by AALP. Now referring to FIG. 3 E, the negative of the gel photograph was scanned by laser densitometry (LKB2222-020 ultrascan XL, by Pharmacia). The percentage surface area under the peak of each band for TNF-α mRNA was normalized to the corresponding GAPDH band.

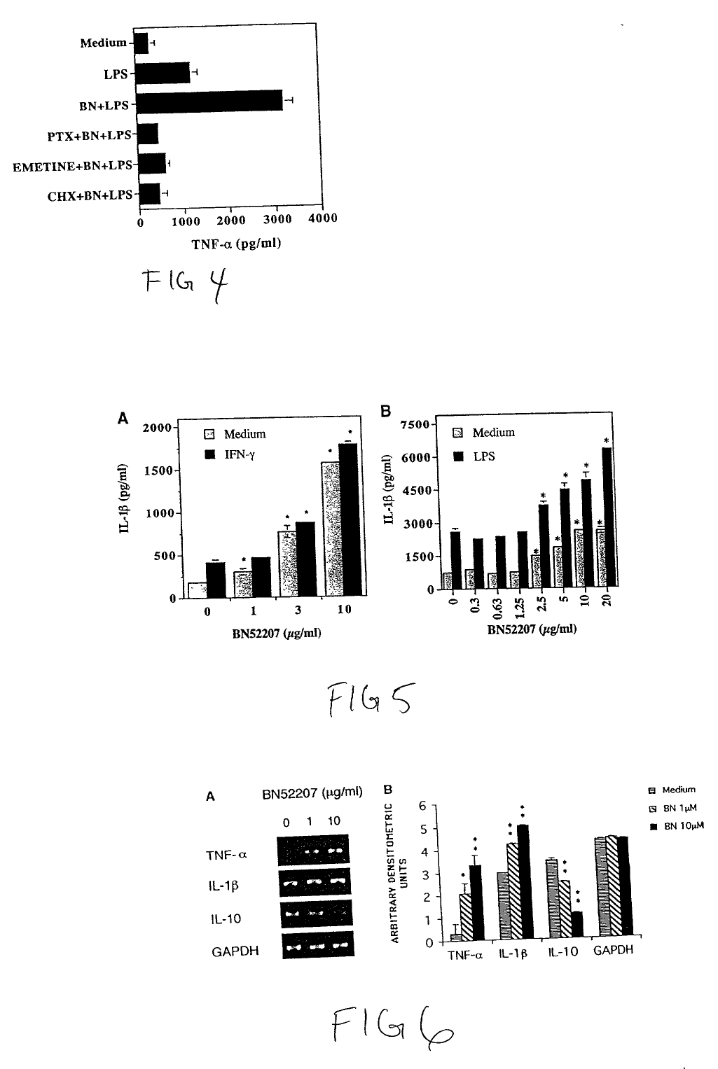

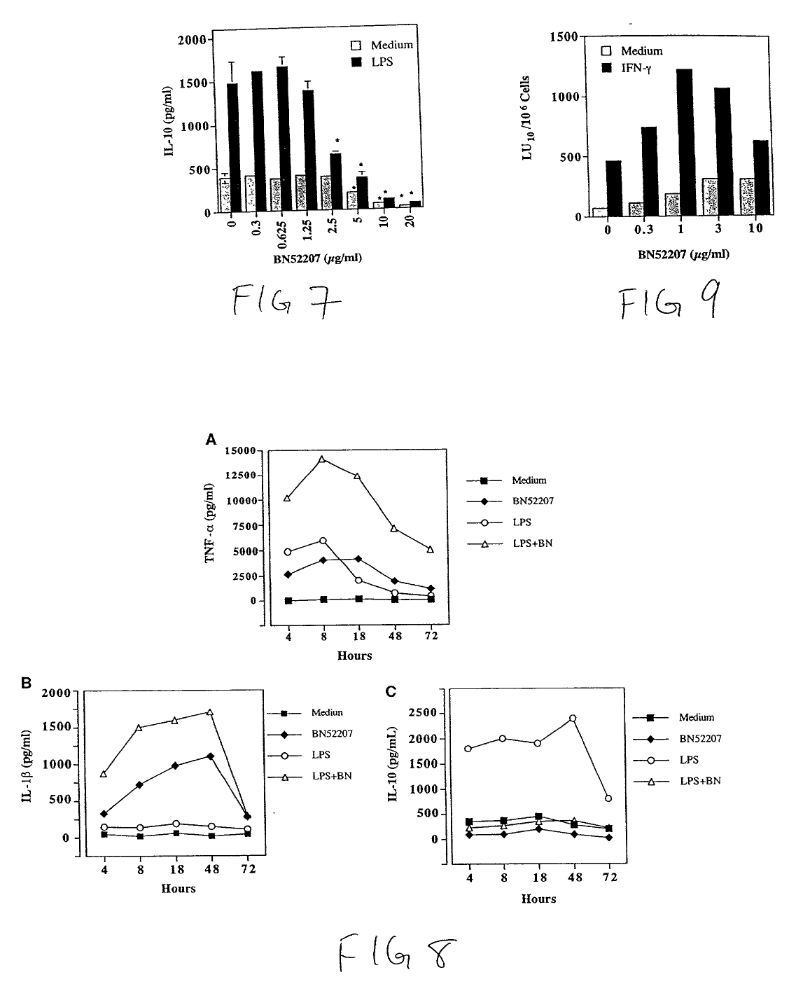

BN52207 potentiated the secretion of TNF-α from IFN-γ-activated PBM and LPS-activated PBM. Further, treatment of PBM with combinations of BN52207 and LPS resulted in an augmented TNF-α secretion. The induction of TNF-α mRNA by BN52207 suggests that TNF-α induction by BN52207 requires de novo synthesis. Now referring to FIG. 4, to confirm de novo synthesis of TNF-α mRNA PBM were treated with protein synthesis inhibitors. PBM were incubated for 18 hours with BN52207 (5 μg/ml) in the absence or presence of LPS (1 μg/ml), LPS (1 μg/ml) plus pentoxifylline (50 μg/ml), emetine (1 μg/ml), and cycloheximide (5 μg/ml). The cell-free supernatants were collected and clarified by centrifugation and the level of TNF-α in the supernatants was assessed by ELISA.

FIG. 4 confirms de novo synthesis of TNF-α mRNA by BN52207 and AALP potentiate TNF-α productions by PBM. Treatment of PBM with protein synthesis inhibitors such as emetine and CHX inhibited BN52207 induced secretion of TNF-α. Further, the addition of pentoxifylline (PTX), which is a selective inhibitor for TNF-α synthesis by PBM, inhibited BN enhanced stimulation of TNF-α secretion by LPS stimulated PBM.

Now referring to FIGS. 5 A and B, the secretion of IL-1β by PBM in the presence or absence of IFN-γ, LPS, and BN52207 is illustrated. PBM was incubated for 18 hours at 37° C. with differing concentrations of BN52207 in the absence or presence of IFN-γ (1000 U/ml) (FIG. 5A) and LPS (1 μg/ml) (FIG. 5B). The supernatants were harvested, clarified by centrifugation, and the presence of IL-1β was assessed by ELISA. Potentiation of IL-1β production by the PBM was noted both for IFN-γ and LPS activated PBM treated with BN52207 (FIG. 5B). However, while BN52207 significantly stimulated untreated PBM, IFN-activated PBM were not further activated by BN52207. These findings demonstrate that BN52207 exert selective effect on the secretion of IL-1β by PBM regardless of the state of activation of the cells.

The de novo synthesis of IL-1β by BN52207 was corroborated in experiments examining IL-1β mRNA by RT-PCR (FIG. 6A). It is clear that IL-1β transcripts are increased in PBM treated with BN52207. This induction was corroborated by induction of TNF-α RNA (FIGS. 6A and B). Noteworthy, IL-10 induction was significantly decreased by BN52207 and the decrease was dependent on the concentration of BN52207 used. These findings demonstrate that AALP exert selective immunoregulatory activities on PBM by inducing selectively TNF-α and IL-1β and by inhibiting IL-10. The inhibition of IL-10 suggest that the potent immunosuppressor TH2 cytokine is regulated by AALP and that a shift of TH2 to TH1 can take place.

Now also referring to FIG. 7, PBM were incubated for 72 hours at 37° C. in the presence of varying concentrations of BN52207 and in the absence or presence of LPS (1 μg/ml). Supernatant aliquots were removed and clarified by centrifugation and the level of IL-10 was assessed by ELISA. Untreated PBM secreted low levels of IL-10 and constitutive secretion was inhibited by BN52207 at concentrations ≦5 μg/ml, and IL-10 mRNA synthesis by PBM was inhibited by BN52207 (FIG. 6B). While LPS significantly stimulated IL-10 secretion by PBM, the addition of BN52207 inhibited the LPS-induced IL-10 secretion. The inhibition was complete with BN52207>10 μg/ml. This concentration of BN52207 at 10 μg/ml was optimal for induction of both TNF-α and IL-1β for PBM. Thus, an inverse correlation was obtained between TNF-α and IL-1β and IL-10 in LPS treated PBM stimulated with BN52207. It was also observed that BN52207 did not activate the secretion of IL-6 by IFN-γ or LPS-activated PBM (data not shown). Accordingly, it was determined that BN52207 and other analogues of AALP are endowed with selective stimulatory and inhibitory signals for cytokine synthesis and secretion.

Now referring to FIGS. 8 A, B, and C, the kinetics of induction/inhibition of cytokine secretion by AALP were examined. PBM, untreated or treated with LPS, were cultured alone or in the presence of a concentration BN52207, such as but not limited to 5 μg/ml, and after defined time intervals, aliquots from the supernatant were removed and examined by ELISA. Referring to FIG. 8A, for TNF-α, the kinetics of cytokine induction were the same for LPS and BN 52207, which was detectable at 4 hours, peaked at 8 hours for LPS, and was slightly prolonged for BN52207 where it was still detected by 48 hours and 72 hours. When LPS and BN52207 were combined, there was a significant potentiation of secretion and this was detected as early as after 4 hours of culture. Furthermore, TNF-α remained at plateau level up to 18 hours and declined thereafter.

Referring to FIG. 8B, for IL-β, induction by BN52207 was also rapid, reaching a plateau at 48 hours and declining thereafter. The same was observed with both LPS and BN52207 with significant potentiation of IL-1β secretion. Referring now to FIG. 8 C, for IL-10, LPS stimulated high levels of IL-10 secretion as early as 4 hours and reached a plateau and then declined markedly at 72 hours. In the presence of BN52207, however, there was a complete inhibition of LPS-induced IL-10 secretion and the inhibitory effect was rapid and observed as early at 4 hours after stimulation. Accordingly, BN52207 acts rapidly in both the induction and the inhibition of cytokine secretion by PBM.

Referring now to FIG. 9, induction of cytotoxicity by AALP treated PBM is illustrated. PBM were cultured for 18 hours at 37° C. in the presence of varying concentrations of BN52207 and in the absence or presence of IFN-γ preferably having a concentration of 1000 U/ml. The cells were harvested and assayed for cytotoxicity against ⁵¹CR-labeled cancer cells, and in particular U937 cancer cells, in an 18 hour cytotoxicity assay as is known in the art. Cytotoxicity was calculated in LU/10⁶ cells from the E:T ratios used. One LU/10⁶ represents a specific cytotoxicity of 10% by 10⁶ effector cells. Accordingly, AALP can stimulate cell-mediated cytotoxic activity against cancer cells in general, and specifically the U937 cancer cells. BN52207 increases PBM cytotoxic activity moderately when used alone and significantly when used in combination with IFN-γ activated PBM. The activation of cytotoxic cells in activated monocytes suggest that AALP induces the cytotoxic machinery of leukocytes and thus potentiates this cytocidal effect against unwanted tissues such as cancer cells and virally infected cells.

Accordingly, it is herein shown that AALP exert selective regulatory effects on cytokine synthesis and secretion by human peripheral blood leukocytes and can be used to treat various diseases through immunomodulation. Various cytokines such as the interleukins (IL), interferons (IFN), colony stimulating factors (CSF) and tumor necrosis factors (TNF) are produced during immune, inflammatory, repair and acute phase responses and they control various aspects of these responses. Cytokines, including the TNF, CSF, IFN and IL mediate host defense responses, cell regulation and cell differentiation. For example, these cytokines can induce fever in a subject, can cause activation of T cells, B cells and macrophages, and can affect the levels of other cytokines, which result in a cascade effect whereby other cytokines mediate the biological levels and actions of the first cytokine.

The present invention also shows that AALP can activate the cytotoxic potential of blood cells and thereby exerting direct or indirect cytotoxicity against harmful pathogens (bacteria, viruses) and tumor cells. The cytotoxic activity by AALP may be the direct result of cytotoxic production and the direct activation of the cytotoxic mechanisms that regulate the cytotoxic function of those cells.

The present invention illustrates the ability of AALP and analogues thereof, to selectively modify certain gene products in blood cells/subsets to produce immunoregulatory effects through cytokine secretion and/or inhibition and these gene products that regulate the cytotoxic function of these cells. Therefore, to treat certain diseases having unwanted and uncontrolled over-suppression or over-stimulation of cytokine activity would be to regulate the expression of the relevant or controlling cytokine or cytokines involved in an immune response without eliminating or over-expressing any one cytokine. Such a treatment would not create or aggravate a pathological or ongoing immune response. In this way, pathological immune-mediated effects, such as immunosuppression or autoimmune reactions, can be prevented and homeostasis can be maintained.

For purposes of illustration but not limitation, monocytes/macrophages are known to play a central role in both innate and adaptive immune responses. In innate immunity, the monocytes/macrophages and natural killer cells are the first cells that encounter pathogenic bacteria through interaction of bacterial surfaces with recognition receptors such as the Toll receptors and other Fc receptors. Phagocytosis takes place and cytokines produced by NK such as IFN-γ and TNF-α activate the macrophages for phagocytosis and degradation of the pathogen. In addition to the direct activation of cytokine by bacteria, AALP potentiates the activity of cytokines both monocytes and NK cells that facilitates and potentiates the activation of phagocytosis and cytocidal activity. In addition, AALP-mediated activity of cytokine secretion of monocytes will result in the activation of NK cells that in turn release cytokine like IFN-γ, TNF-α and GM-CSF. Both macrophages and NK release cytokines that regulate the adaptive immune response through activation of APC function and the regulation of TH1-TH2 differentiation. In adaptive immunity, the monocytes act as antigen presenting cells (APC), thereby processing antigens and export antigenic peptides in the context of class I and II MHC. The conversion of resting monocytes to APC is induced by cytokines released by both monocytes and NK cells. Further, the interaction of APC with T cells results in the induction of cytokine production by APC, such as but not limited to IL-1 and TNF-α, which activate T cells for the expression of IL-2 receptors. Further, the APC secrete other cytokines, like IL-6 and IL-10, that play a role in B cell activation and regulation of TH1 and TH2 responses. AALP selectively triggers TNF-α and IL-1 secretion and inhibits IL-10 and accordingly can be used with the APC to stimulate T helper cell response rather than B cell response. Further, since IL-10 has been shown to regulate the TH1-TH2 response, i.e., by inhibiting TH1 cell-mediated immune response, treatment of PBM with AALP will down-regulate IL-10 and would potentiate TH1 cell-mediated immunity and response to treat conditions that are more responsive to TH1 treatments and pathways.

In addition, AALP's immunomodulatory effects can be used to treat conditions which are known to be susceptible to specific cytokines by administering to a subject a pharmaceutically appropriate dose of AALP via a known pharmaceutical carrier. For purposes of illustration but not limitation, TNF-α is known to participate in macrophage-mediated regulation of microbial infection. It is known that exogenous TNF-α either alone or in combination with IFN-γ activated macrophages mediated killing of infectious organisms both in vitro and in vivo. It is also known that treatment of human monocytes with TNF-α activates mycobactericidal mechanisms and TNF-α significantly increased superoxide anion production. It is also known that TNF-α, IFN-γ, and CSF-1 participate in the activation of macrophages or amebicidal activity. It is also known that administration of TNF-α into organisms with malaria (plasmodium chabandi) suppressed the parsitemias. It is also known that TNF-α plays a crucial role in host resistance to infection to Listeria monocytogenes in vivo and that blocking of TNF-α inhibited listericidal activity of macrophages. In addition, synthesis of TNF-α by activated macrophages plays an autocrine role in the activation of nitric oxide, which is toxic to a variety of pathogens. Therefore, activation of TNF-α secretion by AALP is beneficial in fighting microbial infections and other conditions in which TNF-α either sensitizes the infectious cells and/or organisms, or is a cofactor in elimination of the infectious cells and/or organisms.

In diseases that are dependent on IL-1β production, AALP can be used to potentiate IL-1β secretion. For purposes of illustration but not limitation, it is known that IL-1β potentiates anti-tumor activity. Accordingly, AALP can be used to induce cells to augment IL-1β production in order to treat cancer cells.

In diseases that are dependent on IL-10 production, AALP can be used to inhibit IL-10 secretion. For purposes of illustration but not limitation, parasites are known to increase IL-10 production and thus AALP can be used to inhibit secretion thereof. In addition, B cell lymphomas are known to secrete IL-10 which is involved in cell growth and/or proliferation and resistance to treatments. AALP can be used to inhibit IL-10 secretion and arrest cell growth and to sensitize the defective cells or cancer cells to treatment.

In addition, there are several similarities between the immunomodulating activity mediated by PAF and by AALP. TNF-α production by LPS-treated monocytes is enhanced by PAF. PAF also activates secretion of IL-1 by monocytes. The PAF antagonist BN52021 blocks TNF-α augmented production by PAF in monocytes, whereas petussis toxin partially inhibits the effect of the highest PAF concentration, suggesting mediation through an N-type guanine nucleotide regulatory protein.

Another important factor of the ALP reported here is the direct activation of anti-tumor cytotoxicity by PBM. Thus, AALP are potent sensitizers for cytotoxicity and thus can potentiate the cytotoxic potential of various immunotherpeutic interventions and for vaccines.

The following examples further illustrate one preferred embodiment in practicing the present invention and, of course, should not be construed as in any way limiting its scope, but rather providing at least one preferred embodiment for practicing the same.

### EXAMPLE 1

Five AALP compounds (BN52205, BN52207, BN52211, BN52218, and BN52227) were synthesized at Institut Henri-Beaufour and their chemical structures are illustrated in FIG. 1. The compounds were dissolved in alcohol and stored at 4° C. At the time of assay, the dissolved BN compound were directly diluted at the desired concentrations into RPMI 1640 medium supplemented with 5% BCS, nonessential amino acids (0.1 mM), sodium pyruvate (0.11 g/L), L-glutamine (2 mM), penicillin (100 μ/ml), streptomycin (100 ..g/ml), and amphotericin B (0.25 μg/ml) (complete medium). All the reagents present in the complete medium were purchased from Gibco (Grand Island, N.Y.). The control medium contained the final dilution of alcohol present in the BN-containing medium. Purified human rTNF-α at a specific activity of 76.6×10⁶ U/mg and recombinant interferon-γ (rIFN-γ) at a specific activity of 2.55×10⁷ U/mg were a gift from Genentech (San Francisco, Calif.). The recombinants IL-1β, IL-6, and IL-10 were purchased from PeproTech (Rocky Hills, N.J.).

### EXAMPLE 2

The human premonocytic cell line U937 was obtained from the American Type Culture Collection (Bethesda, Md.). This cell line is sensitive to lysis by NK cells, macrophages, and rTNF-α and was cultured in RPMI 1640 complete medium.

### EXAMPLE 3

Human peripheral blood was obtained from normal volunteers as per HSPC guidelines of UCLA. Peripheral blood mononuclear cells were isolated by FicollHypaque density gradient centrifugation as described previously. These cells were then allowed to adhere to plastic for 1 h at 37° C. in the presence of 5% BCS. The nonadherent cells were pipetted out and the monolayers were washed three times with PBS. The adherent cells, after detachment by scraping, were washed once, resuspended in complete medium, and adjusted to the desired concentrations according to different protocols. The adherent cells prepared by this procedure were primarily monocytes (>90%) as determined by esterase staining and flow cytometry using markers for monocytes, T cells, B cells, and NK cells.

### EXAMPLE 4

Detached PBM were incubated for 18 h in polypropylene test tubes at 2×10⁶ cells/ml in complete medium and AALP analogues were added in the range of 1-30 μg/ml. The culture supernatants, after being clarified by centrifugation, were then harvested and frozen at −70° C. until further use. The PBM were then washed twice with culture medium for assessing direct cell-mediated cytotoxicity.

### EXAMPLE 5

For labeling, the U937 target cells were incubated overnight in 10 ml of complete medium containing 0.1 mCi sodium ⁵¹Cr. Thereafter, the cells were washed three times and adjusted to 10⁵ cells/ml in complete medium. The cytotoxic assay was conducted in triplicate in 96-well U-bottom microliters plates (UV radiated for 15 min). One hundred microliters of PBM at different cell concentrations was added to the plate and was followed by the addition of 5×10^{3 51}Cr-labeled U937 cells to establish different E:T ratios and incubated for 18 h. The specific cytotoxicity was calculated as:

% cytotoxicity=(test cpm−spontaneous cpm)/(total cpm−spontaneuous cpm)×100

Lytic units (LU₁₀/10⁶ cells) were calculated from the E:T titration curve using an exponential fit protocol. One LU₂₀ indicates the number of effector cells necessary to kill 10% of the target cells.

### EXAMPLE 6

The measurement of cytokines in the supernatants was done by ELISA as described previously in the art. The ELISAs were performed in 96-well plates coated with monoclonal antibodies directed against each cytokine: the antibodies were obtained from Dr. Trinchieri for TNF-α; for IL-1β and IL-6, the mAbs were purchased from GENZYME (Cambridge, Mass.), and for IL-10 the mAb was purchased from PHARMINGEN (San Diego, Calif.). The secondary anticytokine specific antibody was prepared in rabbits in our laboratory and was partially purified by an ammonium sulfate cut. An alkaline phosphatase enzyme-linked goat anti-rabbit IgG (CALTAG, South San Francisco, Calif.) was used as the last antibody.

### RT-PCR

RT-PCR was done as previously decribed in the art. Briefly, total cellular RNA was isolated using the guanidinum-isothiocyanate/phenol method or the RNA STAT-60 (TEL-TEST, Inc., Friendswood, Tex.) Following the manufacturer's instructions. The cDNA's were synthesized from 1000 ng of hexamer-primed RNA templates by incubation with MMLV reverse transcriptase (GIBCO, Gaithersburg, Md.) and 1 mM dNTP at 42° C. for 15 min, 99° C. for 5 min, and soaked at 5° C. min. The PCR mixture for each cytokine consisted of 10 mM Tris-HCI, 2 mM MgCl2, 2 mM dNTP, 0.2 μM of up-and downstream oligonucleotide primers, 2.5 μM Ample Taq DNA polymerase (Perkin-Elmer Cetus), and an amount of cDNA equilvalent to 200-250 ng of total RNA. Test aliquots were amplified were amplified by 25-30 cycles of denaturation at 94° C. for 45 s and annealing extension at 55° C. for 30 s. The PCR products were analyzed by ethidium bromide gel electrophoresis, and arbitrary intensity units of the bands were determined by Scan Analysis software. The primers used for PCR amplification were synthesized from published sequences with upstream primer 5′-CAG AGG GAA GAG TTC CCC AG-3′ and downstream primer 5′-CCT TGG TCT GGT AGG AGA CG-3 FOR TNF -\acute{α}; 5′-AAA CAG ATG AAG TGC TCC TTC CAG G-3′ and 5′-TGG AGA ACA CCA CTT GTT GCT CCA-3′ for IL-1β; 5′-CCA ACA GAA GCT TCC ATT CC and 5′-CAC CGG TCG AAC AAT AAA TAT TG-3′ for IL-10; 5′-GAA CAT CAT CCC TGC CTC TAC TG-3′ and 5′-GTT GCT TAA ACC GAT GTC GTT G-3′ for GAPDH.

While there are specific concentrations of agents set forth above, it is to be understood that varying concentrations of the agents can also be used. Therefore, the invention is not limited by the specific concentrations listed above. Furthermore, it is to be understood that biological response modifiers, including specific antibodies, that are known in the art and have immunoregulatory and antiproliferative functions, may also be used in combination with the agents listed above to augment cytotoxicity and/or augment and/or inhibit cytokine production.

While the above description contains many specificities, these should not be construed as limitations on the scope of the invention, but rather as an exemplification of one preferred embodiment thereof. Many other variations are possible without departing from the essential spirit of this invention. Accordingly, the scope of the invention should be determined not by the embodiment illustrated, but by the claims and their legal equivalents in the non-provisional application.

What is claimed is:

1. A method of regulating cytokine activity in a subject having a condition comprising administering the subject an effective amount of cytokine immunomodulatory agent, comprising a glycerol derivative.

2. The method of claim 1, wherein said glycerol derivative is selected from glycerol derivatives of general formulae 1a, 1b, and 1c: 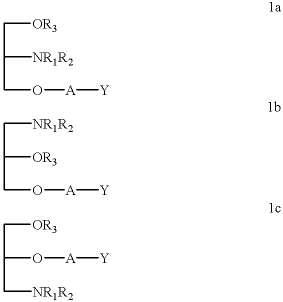
wherein R1 represents a hydrogen atom or a lower alkyl group up to C5;
R2 represents a straight chain or branched chain alkyl group having from 10 to 24 carbon atoms;
R3 represents an aryl or an alkyl radical, CONH-alkyl, CON-dialkyl, each alkyl group having from 1 to 6 carbon atoms;
A stands for: 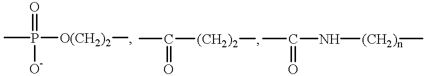
n being an integer of from 2 to 10;
Y represents the following quaternary ammonia: ammonium, alkylammonium, dialkylammonium, trialkylammonium, each alkyl group having from 1 to 6 carbon atoms, or a saturated or unsaturated heterocycle containing nitrogen atom as hetero atom.

3. The method of claim 1, wherein said immunomodulatory agent is an aza-alkyl lysophospholipid.

4. The method of claim 1, wherein said immunomodulatory agent is an analogue of aza-alkyl lysophospholipid.

5. The method of claim 4, wherein said aza-alkyl lysophospholipid is selected from a group consisting of BN52205, BN52207, BN52211, BN52218, BN52227.

6. The method of claim 1, wherein said cytokine activity is TNF activity.

7. The method of claim 1, wherein said cytokine activity is interleukin activity.

8. The method of claim 1, wherein said cytokine activity is interferon activity.

9. The method of claim 1, wherein said condition is an inflammatory response.

10. The method of claim 1, wherein said condition is cachexia.

11. The method of claim 1, wherein said condition is selected from a group consisting of at least a patho-immunogenic response, a response to an antigen, and a response to a vaccine.

12. The method of claim 1, wherein said condition is adult respiratory distress syndrome.

13. The method of claim 1, wherein said condition is a tumor.

14. A method of affecting cytokine activity in a subject having a condition characterized by altered or aberrant cytokine activity, comprising administering to the subject an effective amount of a cytokine regulatory agent, comprising a glycerol derivative is selected from glycerol derivatives of general formulae 1a, 1b, and 1c: 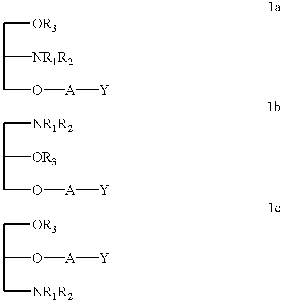
wherein R1 represents a hydrogen atom or a lower alkyl group up to C5;
R2 represents a straight chain or branched chain alkyl group having from 10 to 24 carbon atoms;
R3 represents an aryl or an alkyl radical, CONH-alkyl, CON-dialkyl, each alkyl group having from 1 to 6 carbon atoms;
A stands for: 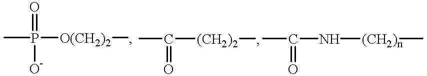
n being an integer of from 2 to 10;
Y represents the following quaternary ammonia: ammonium, alkylammonium, dialkylammonium, trialkylammonium, each alkyl group having from 1 to 6 carbon atoms, or a saturated or unsaturated heterocycle containing nitrogen atom as hetero atom.

15. The method of therapy of claim 14, wherein said cytokine regulatory agent is an aza-alkyl lysophopholipid.

16. The method of therapy of claim 15, wherein the aza-alkyl lysophospholipid is selected from a group consisting of BN52205, BN52207, BN5221 1, BN52218, BN52227.

17. The method of therapy of claim 14, wherein the cytokine regulated is selected from a group consisting of tumor necrosis factor, interleukin, and interferon.

18. The method of therapy of claim 14, wherein the condition is selected from a group consisting of an inflammatory response, cachexia, patho-immunogenic response, antigenic response, vaccine response, adult respiratory distress syndrome, and neoplasia.

19. The method of therapy of claim 16, wherein the cytokine regulated is selected from a group consisting of tumor necrosis factor, interleukin, and interferon.

20. A method of enhancing cytokine activity in a subject having a condition characterized by altered or aberrant cytokine activity, comprising administering to the subject and effective amount of an aza-alkyl lysophospholipid selected from a group consisting of BN52205, BN52207, BN52211, BN52218, BN52227
